SEQUENCE LISTING (5 sequences)

SEQ ID NO: 1 - DNA, 21725 bp, Human
tgcatttcagtaagactcaaatcctccccaccctgggaggcgcagaaagccaaacttcctccaaaaaaaaaaaggcaaaaaaaaaaaaaaaatcacttcagtccaggctgggggtatctggctccccgggaggcgcccgggctccccggggccctggcagacgccggcagcttttctgggcccgcactcggggacctcgcccgcccggcatcgcgattgtgcaagcgccgggcggcaaccacggccgccgctgccggctcctgcccgccgggggagggcggggaggcgcgttcgaagcacacccggtgaactccgggcttcgcatgacttcctgggggttatctccggggtggagtctgccgcccccaccccacctcccgcgcccagcgaacatagccccggtcaccccaggcccggacgccctcgctcacctcgctgactccatcctccttccaccccccctcccctgggtcccgcgtccctcggagtctggccagccgggggccactccgccctcctctgcgtgctcattggccacccagggcatgctctgtctccataaatgcatggtccctgggcataggaacatggagagggacaggaggaaacgcagcgccagcagcatctcatctaccctccttgacacctccccgtggctccagccagaccctagaggtcagccttgcggaccaacaggaggactcccagctttcccttttcaagaggtccccagacaccggccaccctcttccagcccctgcggccagtgcaaggaggcaccaatgctctgaggctgtcgcgtggtgcagcgtcgagcatcctcgccgaggtcctttctgctgcctgtcccgcctcaccccgctccatcacaccagctggccctctttgcttccttttcccagaatcgttaagccccgactcccactagcacctcgtaccaacctcgccccaccccatcctcctgccttcccgcgctccggtgtcccccgctgccatgagctcccccatcagcaagagccgctcgcttgccgccttcctgcagcagctgcgcagtccgaggcagcccccgagactggtgacatctacggcgtacacgtcccctcagccgcgagaggtgccagtctgcccgctgacagctggtggcgagactcagaacgcggccgccctgccgggccccaccagctggccactgctgggcagcctgctgcagattctctggaaagggggtctcaagaaacagcacgacaccctggtaaaccccttttcgcgcccctgactctctcctccctcttccgccctcccagtggcgctccaaaccctccccgacatgcgcgcctcgggcgcatggtgcgcttcggcgtctgggcagggagggtccccggtcccctaggaggaaggaggcgggaggagggaaagcgcttcgggcacggcggaggatgcgcgctgacaccgcgctgtgcccggccggctgcaggtggagtaccacaagaagtatggcaagattttccgcatgaagttgggttcctttgagtcggtgcacctgggctcgccatgcctgctggaagcgctgtaccgcaccgagagcgcgtacccgcagcggctggagatcaaaccgtggaaggcctatcgcgactaccgcaaagaaggctacgggctgctgatcctgtgagtccaatccgagggacctgggcatgcggccagacctgatgagcctgacggcgcctggaaattgtgaagaggcggagttagagttccctggggttggaagcttcttacgtagggcggataccaaagcccctctcccgtgccatcgcaaccctgagctcttgtacagattagagtcggtccacggaaaagccgtgctgtgcacccagcacctgcgttctctctggcaacagggcagggccacaccctcccatcaaagaaacgccaaaacagggttgtaccaactgtggtgttcagactcttgggcaggatgaggttctgaagtcagcgtaccaggcaaacatcttatacaaacaaaattatcccttaaaactccctgaaacaccttgtaagctcaaaagcgaggagttagcctctttggctttttacagtgacattttctgcagaggattgggggacatgccttatttctacttgtaaaggtggaataaggagggatatcaaagttgtgttttaggtcgacaaatgctactatgggatgtgattcttctgctttttagttgttagcatttaacaagttagcacctttatttacccccaaactgtaatgcctaatcgttatattttccaggatataatgactggaattaaaaaccctattaattcacggtgcttcacaggctgtatggtctgctatggggcaggtgcctacaagtgcacgatcgcaaaaggctttttattactccagtgactctctgactgtatgttattatttggaggcgatggcaaaattctaaatctgagatattttgtaaatatgaagcccacaccaatgagttgaaagaagacaagattcttccctcaaacagatctgagttgctggtgatggggtgttcacatgcccccttttaccgctaggaattgtccccaggcctcccagccccactccctgcaaggacacctcaaaatccctgaacccaaattatctggacttcaactttttggtgcaatctttgactaggaggaaaggtgctttttcatcctattgtcaaactattgtcaaaagatgatcactattttatcccaaatattctgatgctaatattctctggctatttcttggcatgattctcagtgtttgtttgattttatggttcctgaagttgcagctggagtatttctgcatctccttcttttcttttcagtgtgctctaaatgtattcctgcttctctcacagggaaggggaagactggcagcgggtccggagtgcctttcaaaagaaactaatgaaaccaggggaagtgatgaagctggacaacaaaatcaatgaggtctgcaggctggggccagctttcctggggctcgtggggacatagggatattggtggagcttcaagtcacagctacaaagaacaaaggaaaaaagaatgcaattcgggtgattcaaagtgggggtcttctctttctctccattccttcctacagcctcctccccaccaaacccttatttctccctcctccaccctcttttaaagtcatatcacaatgagggagcaatggagagtgtggactttggtattctacagccctggatttgaaccccagctttccactccctggctatgagacctggctatataccacttaccttccctaagcctcagtttccccatctacaaaatgggagtaacagttcccaccccacgggtttgctgtgagaattaaatgagataacattgtaaagttcttaagtagtgatatttgagctgggttttgcaggaggagtctgaaagcagagaaagtgggggaaggcattaaggcagacagagtagccttctgaaaacagggaggcatgaaatgacgtggtgtgttcagagaatcggtcatttagtgtagctgatgtgaggctttctctgaaatgatactggataggtcttaacaatatttttgtattatactaaggtatttggacattatcccttagccagcacggagtcaaagggagttttaagcataaagtaatctctttttacttctgttcactttggatgtttattttgaactggatgcgtccccagatgggatcatataggtgaaggattgcaaactcagaagcttccagggctcacatgactaatataaaataataaggcagacccagtaaaaatttcaggaaacgtgatagtgcatgcccctcccaggagggacagcagccccagttccagcaaacgaatgccatgtcagaatgcagacctcatgtcaccaagtctcataattattatttacatttttaaactagtagtggtaaatccatatgttcatatgaaacctcttgttttttttaatggtaacaaattcaaagaacttttaaatgcgctatgcgggcaaattaaacttgcaaactaaatccacacatgtgggctgtatggccacaccagctcacggcagggtccaggatggcaacttatcagctctgtgtgcataagtgagtgacttggcctctttaagcctcagctttctcatctactacatgggaaatgagtagagacggtacctactactacttggttgtcagaggatcaaccaatacacttcgtacagtgtttggtaccatatggacctcattaaatgtgaggcattagttattgaaaactctggtttatgaaactggcatccaaaggtcttatgtgactatcccaaggtcatagactacttagggaaggagctgtgtccagaattggtagatcttctggttccaagttcaacatgcttaacataaccaccaaccaataaagagacgtttgcttgcccagatgtggaagggcacacctcaggatagctgctgcaacacaatccttgagatgctgggccaacagggacagttagctgattcctttgtggttttattcccccattctgattagaaaaaaaaaatgttttccttttcttaaattgccagaattatttttagatcaaaagctgaaacttaagcttcagggtggcttcaggaaaagctcaggaaataacccaggacgaggtcattaggggataaggtgggttcccagggagaatatataattagtgtgacttcaggatgaataacattatggccagtgaatatgcgatgtagcaagacctgaagttagctcagtgagctttaaagttttgttttccttcaacggctttaaaatgtgtcttttgcggttgttttctttgaaggtcttggccgattttatgggcagaatagatgagctctgtgatgaaagaggccacgttgaagacttgtacagcgaactgaacaaatggtcgtttgaaagtaagttgtcgtcacagacttgctcactgattttgcaggtgattttgcggataaaacatggctcttttgacaactcttttgtaaacaggcattattgctgcctagggaaaggccagagcagcccttttaatgcttcttttaggtgcttaagttatgccttgaactatggattgttaatgagtcatacaacaaacatttgttgaatacttgctgtgtgtctggcactgtgctaggtactagggatatgacaatgaatgaaacgcataacccctgtacccttggggagcttatatgctagacaaggtgacagtcaacaagtaaacacattttaaaatatactcttccaaggagtaaaggaaaagagaaagggagttaagaaagaacatgaaaggcaggcatggtggcacatgcctataatcccaggactttgggaggctgaggtgggcagatcgcttgaggccagcccaggaatctgagaacagcttgggcaacacagcaaaaccttgtctctacaaaaaatacaaaaattagtcagggttggtggcacatgcctgtagtcccatctacttgggaggctgaggtgggaggattgcttgaccccgggacgctgaagctaaagtgagccgtgattgtaccactgcattgcagtctgggtgacagtgtgagaccccgtctcaagaaagagagagagacagagaaaggaaggaaggaaggaaggaaggaaggaaggaaggaaggaaggaagaagggagggagggagggaaggaaggaaggagggagggagggagggagggaaggcagaagggagggagggaaggaaggaaggagggagagaaggaaggaaggaaggagggaaggaaaagaaaaagagaaaggaaagaaaaaagaaaagaaataaagaagaaaagagagagagagaaagaaataacatgaaagaatgaaagaggggcaagtcttttttagtaagaagacatttgaggtaagtcctaaaggaagggaaggagcaaatcatgagggtacctgtggaggattgttccaggcagcagagaagaaagtgcaaatgccaggaggtggaagcatgagcgccgagtttgaggaacttcaagctgctggtgtgagtggatcacgatgaacaagctgggaggaaggggagcctgtgaaaggagatgaggtcaaaaagaaggcaggtgtccaacagggcacagctcataggtcctggcaagactttacctctcatctgactcagatggggcaagtaagggagggaggacatgaccgctttcttcaaagattactctggaaataaagggcagagctcactgcccatgggggtgcaagaggagagcaggcagctgtgatcaggtgggacagcgcctggctgaggacattcagggctgtgggagaatctgagagggccacctaagccggacatggccaggggtcgcttcacagagatggtgcccctcagctaagtcctgatggttgagtaggagttcaccaggtgaaaagggaggaaggagagtgtccctgcagaagaagtgcggaatgacaagcatatcctgtaacagaggctggcagagggccagcagccagctcatggagggctgtatgagcgggagtttgaattttagcctgtaagtaaccgggagctgcgggaatgtattcagcagcagtgtgatatatgggcagatttactgggaggatcactgtggctgctgatgggagggatagaaggaaaacctataacacttccataaattcaaggtcagaaatattgccataaatagataaatataagtaactttttttaattacttatttgagacagactctcactctgccccaggctggagtgcagcggcgtgatctcggctcactgcaacctccacctcctgggttcaagcgattctcctgcctcagcatcctgagcagctgggactacaggtgcacaccaccatgcctggctgatttttgtgttttttagtacagatggggtttcaccatgttgcccaggctggtctcgaactgctgacctcaggtgatccacttacctcagcctcccaaagtgctggtattgcaggcgtgcgataccacgcctgactaacttttgtgttttttaagtagagatggggttttgccaccggttggtctcaaactcctgacctcagatgatgctcccacctccacctctgagagtgctgggattacaagcatgagccattgtgcccagcagagtaactaattatttttaaagttcatgtaccatccagagctatctcacatattcccagtggtgtacctgccaaccactgtggaacacaaatacggggaaaccttgaaaggctgggaagaaataagtagagtttggaaaacatggaggtagttggaaaagctgtgggtgggtgctgactaagctaattgactctcactcctcaggtttatgattttaggccatttgtttacctttttaatgctagtctcctactttataagtcaagagccaattatccaccactaaaaagttggttgcagtaatcctgtataattttttttttccagactgtgtcactcaggctagggtgcagtggccagtggtatgatctcggcccactgcaacctcggcctcctgggcccaagagatcttcccacctcagcctccagagtagctggaactacaggcacatgtagccatgcctgaataatttttttatttttatttttttgtagagacagcgttttgccatgttgcccaggctattctcgaactcctgggctcaagcagtctgcctgcccgagcctcctgaagtgttgggattacaggtgtgaaccaccacgctcggccctgtgtcctttttttaaaaaaaaaaggttacaagagataatttttaggacaatggggaacattttaaaatgggacacagtagattttttatggaatggtcgctaaggttcttagggatgttaatgacattgtggttatgttggagaatgctttattcttaggacattactgctaaagtatttaacggtgaagtgacctgagagctcaaaattattttcagatgcttcagcacacacaaccacgtatacacttgtatgcatacatacacacaaagagaaagaaatggcagcaagatgttgatgagttcgggggattcaggtattcattttaccattccttcaacttttctctaggtaaaaagttttgaaatgaagagttgagaattttatttgtaaaaaggttccttctaaataaaatgatgaaatggaaggaaaaaaaaccctatatgaagtgctagcccagtacctggcgtgtaatgggtgtttagaacataattattcccttcccttctcttcctctggtgaatggcttccaattcattatgtcaggagatagaatccaataagtgactctaaaaacacagctgggttagaggcagagtgcagacccgaccctgcagcacctttgctagggcccctggatctcctgtgtgacttgatcaggacagtggatctctgatcatgacactctgaggtgatgagtcattggacctggagataatttctagaaaaagaaagtgatggtgtcccctttctctcagactctgcttttcctcaatattcttggactgttgccttttctcagaaaatcccacacttttaagaggcatagaattgagtctttaataaccagctaaacatagactccaactccagcattgctgtttgtgtaaagtgatgattattttaaatgacaggtatctgcctcgtgttgtatgagaagagatttgggcttctccagaagaatgcaggggatgaagctgtgaacttcatcatggccatcaaaacagtaagcagcctctttaagaaagcagttttcttgattgatgttttacagatcgatttttacagaacttcacagtgattcccaaaaaatggcatacacacatattttatttcaatagttaagtgttcattttaatgtgcattatatattttagtatatttatgtgcatatatgcggctgtctccaacctttttggcaccagggaccagtttcgtggaagacaatttttccatggacagggcagggtggtggggcaggggagggaggatcatttaagtgcattacgtttactgtgcactttatttctgttattattatatactcaccataatgtagaatcagtgggagccctgagcttgttttcgtgcaacacgaaagtcccatctgggggtgatgggagacagtgacagatcatcaggcattagattctcataaggagtgtggaacatagattcctcgcataggcaattcacaatagggttcacactcctgtaagaatctaatgcagccattgatctgagaggaggcagagctcaggtggtaacacctgctggcccgctgatcacctcctgctgcgcggccccgttcctaacaggccactgaccagtactggttcacggcctgggggttggggacccctggatatatgcacgtaacataaagaagcacattaaatccattatttctttgtttaggcaaggttaaaatagctattttaattaaagatcagttgattaaaaatactttttaaaaagagcccaaaatttttcagattttatctttttgaatagtttgggtggcgtgtagataggataaacgctgtgaaattggtttggaaatcactgaagtttggaaattattagaaattgaagtgaatatgaaagttatcgtttttcctctttttttttttttttttacaattaggtcataagattacaatttttatcaaacacatccagtggaaaatcatcccccacattaccttcaatattgaaaggaggtggaggtggggtacgggcattgctcccaggaccggtgcagagagagccgggggtgtccttgtaattccagccttttaaaaagtgcagggtgctgcggtttagaatgttcctcttccagaacgaacattgtctttgagagaaggagaattcagaataaaaaggcatttgcatttctctcagatgatgagcacgtttgggaggatgatggtcactccagtcgagctgcacaagagcctcaacaccaaggtctggcaggaccacactctggcctgggacaccattttcaaatcaggtaaagggccgcccttcacaaccagccagaaccagcccagcacccccgtgtctggagcttcagagaggaggtctggagtgatctcctgagcctcatcaccacatttaggttttgcaaacacaggtgttttggggattgccccagcatacacaaattctaagtaacaagctttctgtagtcttctttgggaattaagagtggttaaaagtaaaatctggccaggtgcagtggctcacccctgtaatcccagcactttgggtggcccaggcgagcagatcacttgaggccaggagtttgagatcagcccggtcaacacggtgaaacccagtctctactacaaatacaaaaattagctgggcgtggtggtgggcacctgtaatcccagctactcaggaggctgaggcaggagaattgcttgaacctgggaggcggaggttgcagtgagccaagactgagccactgcactccatcttgggaggcagagtgagactccatctcaaaataaaaaagtaaaatttatagagtcagggactcctgagttcaagttcagctctgtcactggttgtggtggcttggccaaatgtgcaatcaccttttcatcctcagttgccttgtctctaaaatggactaataattaataataataataagagaacctacctcaatgtaactgtttctgggtctactacagattacagttaacataaaaacctggcatatcacagtatctccgtggactagatctatttctgccacatgacgaaggggaatatttgatgctaggcattgttcacttacacatttagttaatcctcacaacacacctataaggtaggtccttttattagccctgagaggggtacctgagacacatagaagttagccaacgttcccaagattgcatgctaggtgatggcaaagccaaggtttgaagccagacaaggagcttccaggactcgaactctaactgctgtgccatatgtgatcagcatgattattaccatataatgagcctggtggttgtttgcttaatagtgagggaggtacacgcctagattttatgttataaatattagaaatttttaccattattgtcaacatggatggacactctacttgtgcgtatgtatgtggggtatgtgtgcttagtaatatctgggtccaagttggattctgaaagtgaacccaaatcctaccttcttttacacttttttctcccaagatctaaactcttactactcaaaactatgacctgcagcccagtggcatcagcatcacctagaagcttattaggaatgcagagttccaggcctacccaagccctgattcagattctgcattttgagaagatgcctaggtgactcctatccacattaaagtttgagaagccttgctcatgtcggtattgaaaaatccttgccgacttgacaagaaggcaataatataattagtgtgtgtagcaaattgtcagtgttcctgaaaagactatttagatattgcaagaaggagtttggactgaggttttcttctgtgattttcctcatcccaattttcagctgacttcctctctttcttctctgctctaatagtcaaagcttgtatcgacaaccggttagagaagtattctcagcagcctagtgcagatttcctttgtgacatttatcaccagaatcggctttcaaagaaagaattgtatgctgctgtcacagagctccagctggctgcggtggaaacggtaagggtgggctggatttgagtttgctaaatgaacggtctgtaccatttttagatgacagattttaaatagtaattcatctcattcatttcactgggattcactagcatcaactccacaaaaatgaaaagtctcatactagatgccacggtgcctccgcgcacagcatctatgccacggtgcctcagcgcacggcctcataccagacgccacggtgcctccatgcacgggcttctgtgcttaaactctgggtaggaggagacttcatgtgccccagtgacaaaaagccagagtaggatgtgatgagtgccatgatgtccatgcaaactgctacgggagttcaggagcagtaaagttctctgccctggcaagatacagaatccagatttttcaaaattggaagtattttgttcagttgcacatgttaccccaaagtgcctaactgtggctgttacttactgtaaaaaaaaaaaaaaaaaaaaaaaaaaaaaaaaaaggcatactctctttcaagccaataatatttttatctgcttagaattgtaaacaaagtcccaaagagtctgggacaagtggattttaactgaagcaaaacagctcattgtaaagaccaaaattctgtctctgatgtggccgatactccctaggagatagcagatactaagtgagaccaaccccaaacaaatcctgccataggcattcaggtaggtttaattgacttggatactagttcgagctgtgatgggagtggcgcacgctaacctcctcctaagtccaacatttcatgcaccttaaagtctctaagcaaaatgtaaagcagttgtttattttccatgtgcttccccttctgtgctgtttcttgcctgaagtaaaggtagggttttctctatcaggagatagcgaggttctcatttctcctcctgctccacaaggcggttgaaagtgggaatccatcctcgctcactttcctgttgaactgcttaccctgggtggctccttccagtgatcaaagtacagtgtcacccattattcttttattttttcaactggaacgtgacctctctgagggcaatggcttagagttgtccactgctgtgtttccaggacatagaacagtacttggcatctagtagcggctgatagatacttgttgaataaatgaaggaattcaggaactattgagtgcctactgtatgcctgggactgttccaggttcttgagagaccaccatgaaccagagacaagggttcccactctcatggaacttgtatgcaagtggggaagacagacagtagccatgtaaacaaacaaatgtggtcactttagatagtggtgtggattgtggaggacgccatcaggatggtgtaatatagagagatggagatgattaaagggaagccctctctgagaaagcagtactggagctgactctggaataatgagaagagccagccacagaaagacctcgtggaagagcgctgcaggtagaggaaacggcccatgcaaaggccctggggcagaaagagggatggagctacagaactagtaaatgtggtcagagctgagactggagaattggatgggccatgttagccagagccttgtgagcccagcctgggtaaggcattagatttgattgcacctgtgatgggaagctcttgcagcattttaagcaagggaatcacctgatcatttatgtttttaaaagtgggctctggctggtggatggagaatagacttagccaagagagtgggtacagcaggccagtcaatgagcttggtctgttccatggaggtcaatcaaggatagtttgtgcccctgaggttttccaggccacttcccctcaaatcagctgctgcagcttagcccctgcagttttccctacaggctcagactgccttacgtggctttggtaaaagctacagtcatctgccctttggaaactctatttggtacttgcggtgagggactcactgatatggttaaatctgtttcttttttggtcctggctattctgaaagtcacatttggaaaatcagaaccatttgaaagtgtatctaatttgatggctacaccctcttcctgttttgtaaattagactcttataataatggcatctacagtttatttgcagccgaggctgccagtcctctgacatgaagaagactgccttggaggcatcttcttccaaaaataataaaataaccatcatctctgaatggtcttgcacttggtgctcatttgtgtgagtggcacatggcacttagcaacagcgtcttcatgtctgcggaggtggacacaatgatcattttcaggaaatatttggcttaaattagcaaagtacggcctaaaaaataaatacatatgactactagtggtgaatattatgtagcttaaagctacattggctgtttccaccaatattttttctctagtcattgcctgtgcagaggaaataaaagatgccacttaaaagggtgtgggcctttgtcagaaactgtagctggagtactcagctcaatgtttgaatttcttatttttggtgttggcaccctcgtatgagggctgtttacatgtggttgtaaccctcagtgtaggtagtgcctgtttatttttgtggtgactttttagggtgtggccaaaggaacttttaaatttcagaaatgccatttattgtccatttccactataacattctcttaaagcctctcattagcaacaccctgaggatcaccatccctctctgtttactccatttttatatgaatgttttttatatcaagcaacttttaattaaatgtttattttgagatcattgtagattcacattcagttctaagaaaaaatagaaagggatcccttgcatccgttacccagttttccactaatagtaatgtcctgtctaacgagaaaacgttgtcacaatctgaaaattggcattgatttaacccactaatattctcatttcaccaattttacatgtactcatttgtgttcgaacaattattgcgtcaaactgtattttaggtcaggcacagttggctcatgcctgtaatcccagcacttttggaggcaggcagatcacctgaggtcaagagtctaagaccagcctagccaacatggtgaaaccccatctctactaaaaatacaaaaattagccaggcatggtggtggatgcctgtagtcccagctactcaggaggctgaggcaggagaatcgcttgaacccaggaggtggaggttgcagtgagccgagatagtgccactgcactccagtctgggtgacagagtgagactccatctcaaaaaaaaaaaaaaactgtatttaaatgttctaaagcagctgattaaaggattatactttgattccctcagtaaataatttaaaatatatccttaattgatatgttttatgaaataagtctgcttttttatatattggatttgtctaaaggcgtagctataaggacgcgtgaaagagcttcactgatacatttgcagaataaggtggtgggcatcacctttttttcagctaatagtggtttaatgcagttatttacaaaaactcaagtttatgtccatttggcttgcagacagcaaacagtctaatgtggattctctacaatttatcccgtaatccccaagtgcaacaaaagcttcttaaggaaattcaaagtgtattacctgagaatcaggtgccacgggcagaagatttgaggaatatgccgtatttaaaagcctgtctgaaagaatctatgaggtaagagcctatctctgagccaatataaatgtgttagaacactgttctcgtaaacacaaaacaagtggtcccctactcatgggcggcttcccctagctaattagaaacaattatcacactgcaaaaaaatctttggagccatatttcttttctggcactcatttcctaaatcatttgggcaaattctattaaatcttggaaagccagtgccctcccctttaattcaataatcttgctactctaatgaacgctgtaagcactaattaagataggggcatgattgaaggttatgagatggaggctcacaaaagagctatattattatataataggacacagagaaagctttgcttttgagcctctggtataaaacattttttggaaattctttttacatccttttgggaaaacaaaactcacaacattttaggattagaaggatactgtcaactttgtgaagagtagattggagaaacatagaacaaacatcacgcttcaagggctagcacggaaacatacacattttagtctggaatgaagggggaaaaccaacgttaacagtttactaaacagccttgcactggggttttaactaactcaagaagatgagaggaacaagagatacaattattgagaagaaaaaaacaacttctttaggaagatctgtgatcatgctgcctctttgagtatgcacttgggcagcatttatttaatccaatctgcattcccattgacacttcagagagaacttagactgtgctcacctttacatctttgattttatcttttcaggcttacgccgagtgtaccatttacaactcggactcttgacaaggcaacagttctgggtgaatatgctttacccaaaggagtaagtagaatttgtatattctataccttacaaaaccttggaagatacactttgcaaatattcaccagatctccctagactttgtactggaaaatattcattttgggaatgcagagatggtagagaatagttgacatgttgcatagtatagaattagaattcacggctatggagacagagacctgcattcagtttcatcttttatctgtgttgtcttgagtgagttacataatctctgtgagcctcaacttcgtctgtaaaaggggtttaataacagaatctgctgcgcagggtggttgtgagtatggtgcacaattctaaagccctttacctatttattacctggcacattttaagcactcagcgcatgttggcaaccatcattattataaatttgtggatttggcttttgatttgtaaggtgacaacttgtgtcaattttgccattcaaaggtctcttctggttattttaaatactatgtgtctctgcatgtgtttagacagtgctcatgctaaatacccaggtgttgggatccagtgaagacaattttgaagattcaagtcagtttagacctgaacgttggcttcaggagaaggaaaaaattaatccttttgcgcatcttccatttggcgttggaaaaagaatgtgcattggtcgccgattagcagagcttcaactgcatttggctctttgttgggtaaaacctttaccggtttctgcaattatgttctgtgctgtttcacaaggcagaagaagagaggggtaattctgtttttgtgtgcgtgtgcattacagattgtccgcaaatacgacatccaggccacagacaatgagcctgttgagatgctacactcaggcaccctggtgcccagccgggaactccccatcgcgttttgccagcgataatacgcctcaggtgagcatctaggcggttcattatcctctgcaaggtagtgagaatgacgagggatgagctatgctgggaagtcagccagactggggtttgaatcccagctcacctctaacttgctgggtgaccttgagcagttccctaaactctctacttcctttttttttttcatttgtaaaatgatgacccaaagagagtgtatcacaaaaatttaatatgaataccaagtagttattcctgagcctggcatcaattaagtgttcagtaaatgctagcagctgttcatggtttttgcatgtgaatagtaagggttggcaaactttttccgtaaagggccagttggcaaatattttaggttttatggaccatacagtctttgtcacatcctcttaatactaccttggaacatgaaagcagccacaggcaatttgtaaactaatgagtgagctgatgtcctagctttctattgctgccaccgcaaattactaaaacttagtgagttacacgacacacatctattatcttacagttctataggtcagaagtctgatgaggctcttccaggctaaaattatcgtgttgtcggggctctttctggaggttctagaggaggatctattctcttgctagtttggttgttggcagaatttagttcttcatggttacaggactgaagtctttatttttttgctgctgtcagctgaaggcgatcctcaactttgagagatttcccatattccttggctcatgaaaccttccccaactgaaagccagtaacaatggttggttgagtccctctcacactttgaatctctctttcttcttacattgcttctgtctgacaagatattatccaggatcatgtctcccatctccaagttcataaatttaatttcatctggaaagccctttttttcatataaggtagtatattcacgtgttccagggattagggcattcacattttaggagatctgttattctgcctacaaaggctgtgttctgataaattttataaaaaacaagcggtggactgtatttagccctagggtctaggtttgtgttatgcaaccacatgtaatataaatgtatatatttatcagcctgtattgaacacaatagaatattttttctgcagtatgccattcaatactttttctgacaatagaggagttctcatgttccctccacaacttttgccatgtccatataccaaccaggtgctatttatttcaatactactgagttatttcctttaaataacataagccaaatagcatatgttggcctcgtcctgagtaataatttccttaatgaaattaagagttagtaagtctagttatgttctcctaatagacattacaatgaattcaaaactattgaagtgatcaaaagttagcctaagaaggccctaatttttttttttttttttttttttgagacagtgtctcacactgtcacctgggctggagtgcaatggtgcaatctcagctcactgcaacctccgcctcctgggttcatgcgattcttctgcctcagcctcccgagtagctgggattacaggcaggtgcacaccaccacacccagctaattttttgtatttttagtagagatggggtttcattatgttggccagactggtcttgaactcctgacctcgtgatccacccgcctcggcctcccaaagtgctgggattgcaagtgtgagccaccacccctggtggtggaaataagacatttgtaacatatggctttataggctgacatgtcggtgtttaaacccggattctatcattgtgttcttttagcagcaccttctaggattttcctagaaaaaaatgctgtttgccaatggcaagttaaggagcatttgcttattggtagattatttagagaaacatgcaattacagatgaaatttctgaacaaggcccaaatgaatacattattattcctccaccaagtgtctgatgctattatgaaatttgtcaaattctgagcatcccaaccaacagaactgaatcaagtagggtaaggaaagtattctagctgcattgtgacaccccagccttcagcaaagagcatgcatgtttatcacagtaagccacaccttttaagttttagcattttttctttggctaataatttgtcttgtttaaatacacttctttgatatacaattagggtataccttttttttttttttttttttttttttttttttttttttttttttaccaagaccaaaacagccatttcaacttttctgcaagaatggtgcttatggcttctacgttaccggaaagcaaacttcaaacaccacaccatacaacagcttataaagaatatttaggcccaggtgttgatactttttcttctgcaaaattgttccagaagctgtactgtctttctagatggtggtatttgctaacatcatatccaactcagggaagcggactgagtgctgggatccaaggcattctacagggttcactgctggtttacacttcacctgtgtcagcaccatcttcaggtgcttagaatggcctgggagcctgttctgtcttgcatcttccatgacatgaaagggaggctggcacttgtcagtcaggtagaggttacaaaccgtttcaggccctgcctaccacattcactgtttgaatctttaattcccaagaataagtttacatttcacaatgaatgacctacaacagctaaattttctggggctgggagtaatactgacaatccatttactgtagctctgcttaatgtactacttaggaaaatgtccctgcttaataatgtaagccaagctaaatgatggttaaagttatcaggcctcccatgaaattgcgttcttcctgcattgaaataaaaacattattgggaaactagagaacacctctatttttaaaaggactttaacgaagtcaaacaacttataagactagtgattcactggggcattattttgttagaggaccttaaaattgtttattttttaaatgtgattcctttatggcattagggtaaagatgaagcaataatttttaaattgtgtatgtgcatatgaagcacagacatgcatgtgtgtgtgtgtctgtgtgtgtgtgtccgtgtatgtgtgtgtgggttctaatggtaatttgcctcagtcatttttttaatatttgcagtacttgatttaggatctgtggtgcagggcaatgtttcaaagtttagtcacagcttaaaaacattcagtgtgactttaatattataaaatgatttcccatgccataatttttctgtctattaaatgggacaagtgtaaagcatgcaaaagttagagatctgttatataacatttgttttgtgatttgaactcctaggaaaaatatgatttcataaatgtaaaatgcacagaaatgcatgcaatacttataagacttaaaaattgtgtttacagatggtttatttgtgcatatttttactactgcttttcctaaatgcatactgtatataattctgtgtatttgataaatatttcttcctacattatatttttagaatatttcagaaatatacatttatgtctttatattgtaataaatatgtacatatctaggtatatgctttctctctgctgtgaaattatttttagaattataaattcacgtcttgtcagatttcatctgtataccttcaaattctctgaaagtaaaaataaaagtttttaaatatttgagagacttcccctctttattgcctttgttacaacaaaataaaaacaaattcaaactttattttactggtagaaaattttctcatatggcctacaagccctatatcacaaaatttacccttagataaatctagactttaatagcagttttactgaaagtttattcttgtaattgcaaaataactttccattttgttgctgcttaaatatttcccaagtccagttacttctatctctaccccctttccaatcacacaatcagtcacacactggttgtgtgattgaaaggggggtagaacggcctgggagcctgttctctcttgtatctttcatgacatgaaagggaggctggcacttggcagtcaggtagaggttacgaaccatttcagcccctgcctaccacattcactgtttgaatacttaattcccaagaataagtttacatttcacaatgaaagacctacaacagccaaattttccgtggctgggagcaatactgacaatctcttgcctggaccactaatatctctagtctctgcaggagctcccagctgatctcagtcctctcttgcttccttcccctctgttctttgcacactt

SEQ ID NO: 2 - DNA, 3295 bp, Human
tggagagggacaggaggaaacgcagcgccagcagcatctcatctaccctccttgacacctccccgtggctccagccagaccctagaggtcagccttgcggaccaacaggaggactcccagctttcccttttcaagaggtccccagacaccggccaccctcttccagcccctgcggccagtgcaaggaggcaccaatgctctgaggctgtcgcgtggtgcagcgtcgagcatcctcgccgaggtcctttctgctgcctgtcccgcctcaccccgctccatcacaccagctggccctctttgcttccttttcccagaatcgttaagccccgactcccactagcacctcgtaccaacctcgccccaccccatcctcctgccttcccgcgctccggtgtcccccgctgccatgagctcccccatcagcaagagccgctcgcttgccgccttcctgcagcagctgcgcagtccgaggcagcccccgagactggtgacatctacggcgtacacgtcccctcagccgcgagaggtgccagtctgcccgctgacagctggtggcgagactcagaacgcggccgccctgccgggccccaccagctggccactgctgggcagcctgctgcagattctctggaaagggggtctcaagaaacagcacgacaccctggtggagtaccacaagaagtatggcaagattttccgcatgaagttgggttcctttgagtcggtgcacctgggctcgccatgcctgctggaagcgctgtaccgcaccgagagcgcgtacccgcagcggctggagatcaaaccgtggaaggcctatcgcgactaccgcaaagaaggctacgggctgctgatcctggaaggggaagactggcagcgggtccggagtgcctttcaaaagaaactaatgaaaccaggggaagtgatgaagctggacaacaaaatcaatgaggtcttggccgattttatgggcagaatagatgagctctgtgatgaaagaggccacgtcgaagacttgtacagcgaactgaacaaatggtcgtttgaaagtatctgcctcgtgttgtatgagaagagatttgggcttctccagaagaatgcaggggatgaagctgtgaacttcatcatggccatcaaaacaatgatgagcacgtttgggaggatgatggtcactccagtcgagctgcacaagagcctcaacaccaaggtctggcaggaccacactctggcctgggacaccattttcaaatcagtcaaagcttgtatcgacaaccggttagagaagtattctcagcagcctagtgcagatttcctttgtgacatttatcaccagaatcggctttcaaagaaagaattgtatgctgctgtcacagagctccagctggctgcggtggaaacgacagcaaacagtctaatgtggattctctacaatttatcccgtaatccccaagtgcaacaaaagcttcttaaggaaattcaaagtgtattacctgagaatcaggtgccacgggcagaagatttgaggaatatgccgtatttaaaagcctgtctgaaagaatctatgaggcttacgccgagtgtaccatttacaactcggactcttgacaaggcaacagttctgggtgaatatgctttacccaaaggaacagtgctcatgctaaatacccaggtgttgggatccagtgaagacaattttgaagattcaagtcagtttagacctgaacgttggcttcaggagaaggaaaaaattaatccttttgcgcatcttccatttggcgttggaaaaagaatgtgcattggtcgccgattagcagagcttcaactgcatttggctctttgttggattgtccgcaaatacgacatccaggccacagacaatgagcctgttgagatgctacactcaggcaccctggtgcccagccgggaactccccatcgcgttttgccagcgataatacgcctcagatggtggtatttgctaacatcatatccaactcagggaagcggactgagtgctgggatccaaggcattctacagggttcactgctggtttacacttcacctgtgtcagcaccatcttcaggtgcttagaatggcctgggagcctgttctgtcttgcatcttccatgacatgaaagggaggctggcacttgtcagtcaggtagaggttacaaaccgtttcaggccctgcctaccacattcactgtttgaatctttaattcccaagaataagtttacatttcacaatgaatgacctacaacagctaaattttctggggctgggagtaatactgacaatccatttactgtagctctgcttaatgtactacttaggaaaatgtccctgcttaataatgtaagccaagctaaatgatggttaaagttatcaggcctcccatgaaattgcgttcttcctgcattgaaataaaaacattattgggaaactagagaacacctctatttttaaaaggactttaacgaagtcaaacaacttataagactagtgattcactggggcattattttgttagaggaccttaaaattgtttattttttaaatgtgattcctttatggcattagggtaaagatgaagcaataatttttaaattgtgtatgtgcatatgaagcacagacatgcatgtgtgtgtgtgtctgtgtgtgtgtgtccgtgtatgtgtgtgtgggttctaatggtaatttgcctcagtcatttttttaatatttgcagtacttgatttaggatctgtggtgcagggcaatgtttcaaagtttagtcacagcttaaaaacattcagtgtgactttaatattataaaatgatttcccatgccataatttttctgtctattaaatgggacaagtgtaaagcatgcaaaagttagagatctgttatataacatttgttttgtgatttgaactcctaggaaaaatatgatttcataaatgtaaaatgcacagaaatgcatgcaatacttataagacttaaaaattgtgtttacagatggtttatttgtgcatatttttactactgcttttcctaaatgcatactgtatataattctgtgtatttgataaatatttcttcctacattatatttttagaatatttcagaaatatacatttatgtctttatattgtaataaatatgtacatatctaggtatatgctttctctctgctgtgaaattatttttagaattataaattcacgtcttgtcagatttcatctgtataccttcaaattctctgaaagtaaaaataaaagtttttaaatattaaaaaaaaaaaaaaaaaaaaa

SEQ ID NO: 3 - DNA, 24 bp, Artificial
  RT-PCR primer for CYP24 mRNA
ggactcttgacaaggcaacagttc

SEQ ID NO: 4 - DNA, 24 bp, Artificial
  RT-PCR primer for CYP24 mRNA
ttgtctgtggcctggatgtcgtat

SEQ ID NO: 5 - DNA, 3274 bp, Human
tggagagggacaggaggaaacgcagcgccagcagcatctcatctaccctccttgacacctccccgtggctccagccagaccctagaggtcagccttgcggaccaacaggaggactcccagctttcccttttcaagaggtccccagacaccggccaccctcttccagcccctgcggccagtgcaaggaggcaccaatgctctgaggctgtcgcgtggtgcagcgtcgagcatcctcgccgaggtcctttctgctgcctgtcccgcctcaccccgctccatcacaccagctggccctctttgcttccttttcccagaatcgttaagccccgactcccactagcacctcgtaccaacctcgccccaccccatcctcctgccttcccgcgctccggtgtcccccgctgccatgagctcccccatcagcaagagccgctcgcttgccgccttcctgcagcagctgcgcagtccgaggcagcccccgagactggtgacatctacggcgtacacgtcccctcagccgcgagaggtgccagtctgcccgctgacagctggtggcgagactcagaacgcggccgccctgccgggccccaccagctggccactgctgggcagcctgctgcagattctctggaaagggggtctcaagaaacagcacgacaccctggtggagtaccacaagaagtatggcaagattttccgcatgaagttgggttcctttgagtcggtgcacctgggctcgccatgcctgctggaagcgctgtaccgcaccgagagcgcgtacccgcagcggctggagatcaaaccgtggaaggcctatcgcgactaccgcaaagaaggctacgggctgctgatcctggaaggggaagactggcagcgggtccggagtgcctttcaaaagaaactaatgaaaccaggggaagtgatgaagctggacaacaaaatcaatgaggtcttggccgattttatgggcagaatagatgagctctgtgatgaaagaggccacgttgaagacttgtacagcgaactgaacaaatggtcgtttgaaagtatctgcctcgtgttgtatgagaagagatttgggcttctccagaagaatgcaggggatgaagctgtgaacttcatcatggccatcaaaacaatgatgagcacgtttgggaggatgatggtcactccagtcgagctgcacaagagcctcaacaccaaggtctggcaggaccacactctggcctgggacaccattttcaaatcagtcaaagcttgtatcgacaaccggttagagaagtattctcagcagcctagtgcagatttcctttgtgacatttatcaccagaatcggctttcaaagaaagaattgtatgctgctgtcacagagctccagctggctgcggtggaaacgacagcaaacagtctaatgtggattctctacaatttatcccgtaatccccaagtgcaacaaaagcttcttaaggaaattcaaagtgtattacctgagaatcaggtgccacgggcagaagatttgaggaatatgccgtatttaaaagcctgtctgaaagaatctatgaggcttacgccgagtgtaccatttacaactcggactcttgacaaggcaacagttctgggtgaatatgctttacccaaaggaacagtgctcatgctaaatacccaggtgttgggatccagtgaagacaattttgaagattcaagtcagtttagacctgaacgttggcttcaggagaaggaaaaaattaatccttttgcgcatcttccatttggcgttggaaaaagaatgtgcattggtcgccgattagcagagcttcaactgcatttggctctttgttggattgtccgcaaatacgacatccaggccacagacaatgagcctgttgagatgctacactcaggcaccctggtgcccagccgggaactccccatcgcgttttgccagcgataatacgcctcagatggtggtatttgctaacatcatatccaactcagggaagcggactgagtgctgggatccaaggcattctacagggttcactgctggtttacacttcacctgtgtcagcaccatcttcaggtgcttagaatggcctgggagcctgttctgtcttgcatcttccatgacatgaaagggaggctggcacttgtcagtcaggtagaggttacaaaccgtttcaggccctgcctaccacattcactgtttgaatctttaattcccaagaataagtttacatttcacaatgaatgacctacaacagctaaattttctggggctgggagtaatactgacaatccatttactgtagctctgcttaatgtactacttaggaaaatgtccctgcttaataatgtaagccaagctaaatgatggttaaagttatcaggcctcccatgaaattgcgttcttcctgcattgaaataaaaacattattgggaaactagagaacacctctatttttaaaaggactttaacgaagtcaaacaacttataagactagtgattcactggggcattattttgttagaggaccttaaaattgtttattttttaaatgtgattcctttatggcattagggtaaagatgaagcaataatttttaaattgtgtatgtgcatatgaagcacagacatgcatgtgtgtgtgtgtctgtgtgtgtgtgtccgtgtatgtgtgtgtgggttctaatggtaatttgcctcagtcatttttttaatatttgcagtacttgatttaggatctgtggtgcagggcaatgtttcaaagtttagtcacagcttaaaaacattcagtgtgactttaatattataaaatgatttcccatgccataatttttctgtctattaaatgggacaagtgtaaagcatgcaaaagttagagatctgttatataacatttgttttgtgatttgaactcctaggaaaaatatgatttcataaatgtaaaatgcacagaaatgcatgcaatacttataagacttaaaaattgtgtttacagatggtttatttgtgcatatttttactactgcttttcctaaatgcatactgtatataattctgtgtatttgataaatatttcttcctacattatatttttagaatatttcagaaatatacatttatgtctttatattgtaataaatatgtacatatctaggtatatgctttctctctgctgtgaaattatttttagaattataaattcacgtcttgtcagatttcatctgtataccttcaaattctctgaaagtaaaaataaaagtttttaaatatt

(19) United States
(12) Patent Application Publication
(10) Pub. No.: US 2007/0207488 A1
(43) Pub. Date: Sep. 6, 2007

(54) Method for identifying altered vitamin D metabolism

(76) Inventors: Donald L. Trump, Pittsburgh, PA (US); Josephia Muindi, Willamsville, NY (US); Lionel J. Coignet, East Amherst, NY (US); Candace S. Johnson, Amherst, NY (US)

Correspondence Address: HODGSON RUSS LLP;THE GUARANTY BUILDING, 140 PEARL STREET, SUITE 100, BUFFALO, NY, 14202-4040, US

(21) Appl. No.: 11/700,462

(22) Filed: Jan. 31, 2007

Related U.S. Application Data
(60) Provisional application No. 60/763,565, filed on Jan. 31, 2006.

(51) Int. Cl. C12Q 1/68; C12P 19/34

(52) U.S. Cl. 435/6; 435/91.2

(57) ABSTRACT

A method is provided for identifying an individual as having altered vitamin D metabolism comprising analyzing a biological sample from the individual for the presence of CYP24 SNPs and/or aberrantly spliced CYP24 mRNA. The presence of the SNPs and/or the aberrantly spliced CYP24 mRNA indicates that the individual has altered vitamin D metabolism. Also provided are methods for customizing dosages of biologically active vitamin D compounds for individuals who are determined to have altered vitamin D metabolism.

This invention claims priority to U.S. Provisional Patent Application Ser. No. 60/763,565, filed Jan. 31, 2006, the entire disclosure of which is incorporated herein by reference.

This work was supported by funding from the National Cancer Institute, grant nos. RO1-CA-95045-01, RO1-CA-67267-10, RO1-CA-85142-05 and RO1-CA-112914-01. The Government has certain rights in the invention.

### FIELD OF THE INVENTION

The present invention relates generally to the field of diseases associated with vitamin D and more particularly to determining alterations in vitamin D metabolism in an individual.

### BACKGROUND OF THE INVENTION

Considerable epidemiologic data suggest that vitamin D exposure influences mortality of cancer (prostate, breast, colorectal and lymphoma, melanoma and lung cancer respectively), osteoporosis and autoimmune diseases such as multiple sclerosis. Markers of vitamin D exposure that have been linked to disease occurrence include latitude of habitation, circulating vitamin D binding protein, blood vitamin D levels and vitamin D receptor polymorphisms. However, careful study of these factors provides conflicting data on their power to predict whether any given individual will experience abnormal vitamin D exposure.

With respect to treatment for bone related disorders, calcium and vitamin D supplements are an effective treatment to reduce bone loss in the elderly. Most individuals can obtain adequate calcium in their diet but supplements are an alternative for people who find this difficult. Calcium alone has a limited effect as a treatment for osteoporosis, but combined with vitamin D, it is particularly helpful for the elderly and housebound who cannot obtain natural sunlight and may have a poor diet. Calcitriol is an activated form of vitamin D given to post-menopausal women who have osteoporosis. Calcitriol improves the absorption of calcium from the gut, as calcium cannot be absorbed without vitamin D. However, it is not known if individual differences are present in the absorption and metabolism of calcitriol such that exposure to calcitriol would be affected. Such information would be important for, among other reasons, customizing dosages of vitamin D, as well as its analogs, metabolites. Therefore, there is a need for methods of identifying whether a particular individual is likely to have altered vitamin D metabolism.

### SUMMARY OF THE INVENTION

The present invention provides a method for identifying an individual as likely having altered vitamin D metabolism. The method comprises obtaining a biological sample from the individual and determining the presence of certain CYP24 single nucleotide polymorphisms (SNPs) and/or aberrantly spliced CYP24 mRNA, and/or correctly spliced CYP24 mRNA in the absence of calcitriol, wherein the presence of the SNPs and/or aberrantly spliced CYP24 and/or correctly spliced CYP24 mRNA in the absence of calcitriol is indicative that the individual is likely to have altered vitamin D metabolism.

Also provided is a method for customizing dosing of calcitriol or calcitriol precursors, or a vitamin D analog compound that does not generate as much of a calcemic response as calcitriol. The method comprises obtaining a biological sample from the individual, identifying the presence of CYP24 SNPs and/or aberrantly spliced CYP24 mRNA and/or correctly spliced CYP24 mRNA in the absence of calcitriol, and based upon such identification, prescribing a lower or higher dose of calcitriol or calcitriol precursors.

### BRIEF DESCRIPTION OF THE DRAWINGS

FIG. 1 is a flow-chart depicting steps in the metabolism of vitamin D.

FIG. 2 is a graphical depiction of CYP24 enzymatic activity measured in untreated and calcitriol treated human cancer cell lines. The results show that human cancer cell lines can be classified into three categories based on their baseline and calcitriol-induced CYP24 enzyme profiles. Category I: prostate (LNCaP) and lung (H520) cancer cell lines with negligible baseline and calcitriol-induced CYP24 activity. Category II: prostate (PC3), breast (MCF7) and colon (HT29) cancer cell lines with barely detectable baseline CYP24 activity that is calcitriol-induced. Category III: prostate (DU145), breast (MDA231), lung (A549) and colon (HCT116) cancer cell lines with high baseline and calcitriol induced CYP24 activity.

FIGS. 3A-3D provide graphical representations of CYP24 mRNA splicing patterns and cDNA amplification profiles. FIG. 3A provides a graphical map of the CYP24 gene from exon 9 through exon 11, an example of primer locations for obtaining cDNA using RT-PCR, and resulting products in the case of correct (280 bp) and aberrant (880 bp) splicing. FIG. 3B provides a graphical map of the CYP24 gene from exon 11 through exon 12 and RT-PCR cDNA product sizes for correctly spliced CYP24 mRNA from a CYP24 gene lacking a predictive SNP (150 bp), as well as the inclusion of intron 12 sequences in aberrantly spliced CYP24 mRNA when the predictive SNP is present (302 bp transcript). FIG. 3C is a photographic representation of cDNA amplification products of unspliced or spliced CYP24 mRNA across exons 9-11 of the CYP24 mRNA. FIG. 3D is a photographic representation of cDNA amplification products across exons 11-12 of correctly spliced (150 bp) or aberrantly spliced (302 bp) CYP24 mRNA from various cancer cell lines.

FIG. 4 is a graphical representation showing correlation between rate of serum calcitriol clearance (elimination half life, (T_{1/2} hr) and polymorphisms at positions 15752 and 15774 of SEQ ID NO:1 in intron sequences between exon 9 and 10 of CYP24 gene in 30 patients after oral administration of high doses of calcitriol. TT/TT (at positions 15752 and 15774, respectively, of SEQ ID NO:1) genotype tends to have significantly lower T_{1/2} than the TC/TC genotype, p-value=0.0377 (one-sided, Fisher exact test). The lower the T_{1/2}, the higher is the systemic exposure for a given dose of calcitriol. Normal expression is as expressed from individuals who are homozygous TT. Elimination half life was calculated using the equation: T_{1/2}=0.693/β, where β is the slope of the regression line of the terminal log serum calcitriol concentration versus time.

### DETAILED DESCRIPTION OF THE INVENTION

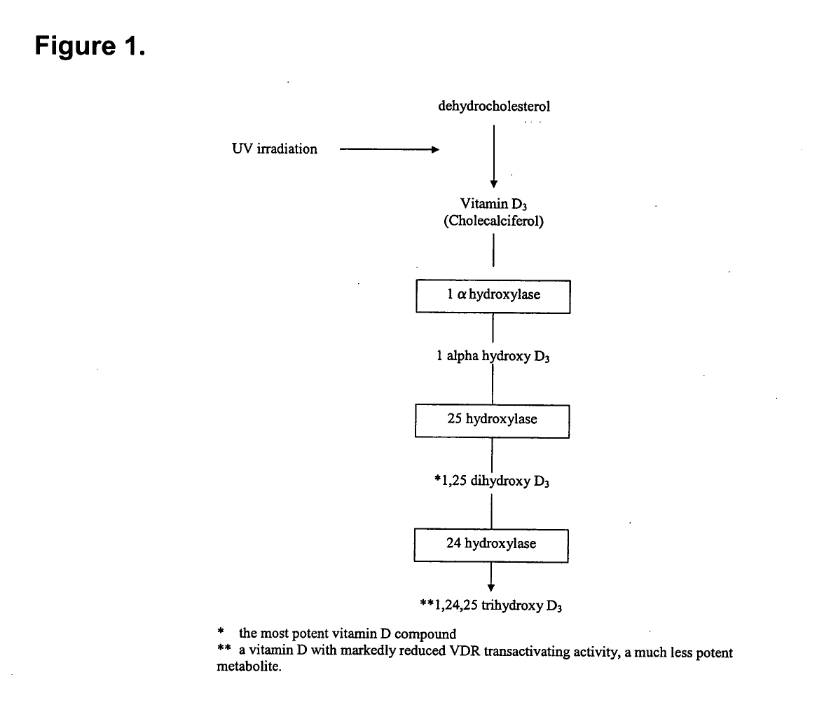

An outline of the steps of vitamin D metabolism is depicted in FIG. 1. The enzymes 1α hydroxylase and 24 hydroxylase (CYP24) are present in kidney and liver, respectively, and are involved in the metabolism of vitamin D and systemic exposure to it and to its metabolites, such as calcitriol. In addition, several types of cells in the body express these enzymes (e.g. prostate) and hence, intracellular synthesis and catabolism of calcitriol may also influence cellular vitamin D exposure in an organ in an enzyme activity-related fashion.

CYP24 is a mitochondrial enzyme that inactivates calcitriol. CYP24 is expressed in forms with varying enzymatic activity in different cells of the body indicating that varying calcitriol exposure at the cellular or organ-specific level may occur and influence disease development.

The terms “calcitriol exposure” and “vitamin D exposure” relate to circulating calcitriol levels over time, particularly after calcitriol treatment. In the present invention, certain single nucleotide polymorphisms (SNPs) in the CYP24 gene have been discovered to be markers of alterations in expression of CYP24 mRNA in the form of splice variants. The SNPs are also demonstrated to be correlated with the expression and function of the CYP24 protein.

Based on these discoveries, the present invention provides a method for identifying an individual who is likely to have altered vitamin D metabolism. The method comprises obtaining a biological sample from an individual and determining the presence of certain CYP24 gene SNPs, and/or aberrantly spliced CYP24 mRNA, and/or calcitriol insensitive splicing, wherein the presence of the SNPs and/or aberrantly spliced CYP24 mRNA and/or calcitriol insensitive splicing is indicative that the individual is likely to have altered calcitriol catabolism. By “altered calcitriol catabolism” it is meant that the individual exhibits a higher or lower rate of clearance of caltictriol from the body relative to the rate of calcitriol clearance from an individual who does not exhibit the CYP24 SNPs, aberrantly spliced CYP24 mRNA and calctriol insensitive splicing. In addition to altered calcitriol clearance, “altered calcitriol catabolism” can be also be evidenced by CYP24 protein from an individual exhibiting reduced enzymatic activity compared to CYP24 protein translated from correctly spliced CYP24 mRNA.

By “calctriol insensitive splicing” it is meant that the predominant form of RNA in a biological sample is correctly spliced CYP24 mRNA, whether or not calcitriol is present before the CYP24 mRNA is spliced.

By “correctly spliced CYP24 mRNA” it is meant that the CYP24 mRNA does not include any polynucleotide sequence transcribed from introns between the DNA sequence encoding exons 9-12 of the CYP24 mRNA.

By “aberrantly spliced CYP24 mRNA” it is meant that the CYP24 mRNA includes at least some polynucleotide sequence transcribed from the introns between the DNA sequence encoding exons 9-12 of the CYP24 mRNA. In this regard, the genomic sequence of the human CYP24 gene is presented as SEQ ID NO:1. The sequence of CYP24 cDNA generated from correctly spliced CYP24 mRNA is provided in SEQ ID NO:2. The nucleotide positions which designate the boundaries of the CYP24 exons and introns (as transcribed into CYP24 heteronuclear RNA) are presented in Table 1. Accordingly, from a comparison of Table 1 and the genomic sequence of the CYP24 gene (SEQ ID NO:1), (as well as from a comparison of SEQ ID NO:1 to the cDNA sequence presented in SEQ ID NO:2), one can easily distinguish nucleotide sequences from exons and introns, and thereby determine whether any particular mRNA has been correctly or aberrantly spliced as defined herein. It will be recognized that nucleotide sequences complementary to the sequences associated with SEQ ID NO's presented herein can also be readily determined if necessary.

TABLE 1

| Exon # | Starts in SEQ ID NO: 1 | Ends in SEQ ID NO: 1 |
|---|---|---|
| 1 | 589 | 1254 |
| 2 | 1476 | 1666 |
| 3 | 2904 | 3001 |
| 4 | 4884 | 4985 |
| 5 | 8741 | 8832 |
| 6 | 10011 | 10121 |
| 7 | 10990 | 11137 |
| 8 | 14690 | 14858 |
| 9 | 15648 | 15728 |
| 10 | 16229 | 16426 |
| 11 | 16524 | 16645 |
| 12 | 19012 | 20325 |

It will also be recognized by those skilled in the art that, while determining whether CYP24 mRNA is aberrantly spliced could be performed by determining the sequence of the mRNS, such sequence determinations are not required. For example, primers can be designed to amplify CYP24 mRNA such that aberrantly spliced mRNA can be readily identified by alterations in cDNA size due to the inclusion of intron sequences in the mRNA. For instance, in one embodiment, a forward and reverse primer can be used in RT-PCR for amplifying aberrantly spliced CYP24 mRNA in the form of an mRNA from which the introns between exons 9 and 11 have not been spliced, and subsequent analysis of the electrophoretic mobility of the amplified RT-PCR products. One example of a suitable primers for this purpose includes a forward primer of the sequence ggactcttgacaaggcaacagttc (SEQ ID NO:3, and a reverse primer of the sequence ttgtctgtggcctggatgtcgtat (SEQ ID NO:4). Using this combination of primers in an RT-PCT reaction to amplify CYP24 mRNA into cDNA from certain cancer cells reveals an aberrantly spliced mRNA sequence of 880 base pairs (bp), while a correctly spliced CYP24 mRNA sequence is 280 bp, due to the lack of intron sequences in the correctly spliced CYP24 mRNA sequence.

It has also been discovered that certain SNPs in the CYP24 gene can be used to determine whether an individual is likely to have altered vitamin D metabolism. For example, a SNP that is believed to cause an aberrantly spliced mRNA that, via RT-PCR, results in a cDNA with the sequence of SEQ ID NO:5, can be identified at position −1 from the beginning of exon 12 in the CYP24 gene (nucleotide number 19011 in SEQ ID NO:1; see Table 2). In this regard, certain SNPs identified herein are shown to be correlated with altered CYP24 enzymatic activity, in addition to being correlated with aberrant splicing and calcitriol insensitive splicing of CYP24 mRNA.

SNPs informative as to the likelihood of an individual having altered vitamin D metabolism are also present in CYP24 introns between exons 9 and 10 and between exons 11 and 12. These SNPs are also presented in Table 2. The normal sequence is TTGG for the SNPs numbered 1-4.

TABLE 2

| | SNP position in SEQ ID NO: 1 (in intron | | | |
|---|---|---|---|---|
| | between exon 9 and 10 for SNPs 1-3, and | | | |
| | in intron between 11-12 for SNP 4) | LNCaP | PC3 | DU145 |
| 1 | 15752 | T | C | T |
| 2 | 15774 | T | C | T |
| 3 | 15876 | G | A/G | G |
| 4 | 19011 | G/T | G | G |

With respect to the cell lines in Table 2, LNCaP is an androgen dependent human prostate cancer cell line sensitive to calcitriol growth inhibition while PC3 and DU145 are androgen independent human prostate cancer cells and are relatively resistant to calcitriol growth inhibition. These cells lines are useful for characterization of SNPs and CYP24 mRNA splicing patterns which alter vitamin D metabolism.

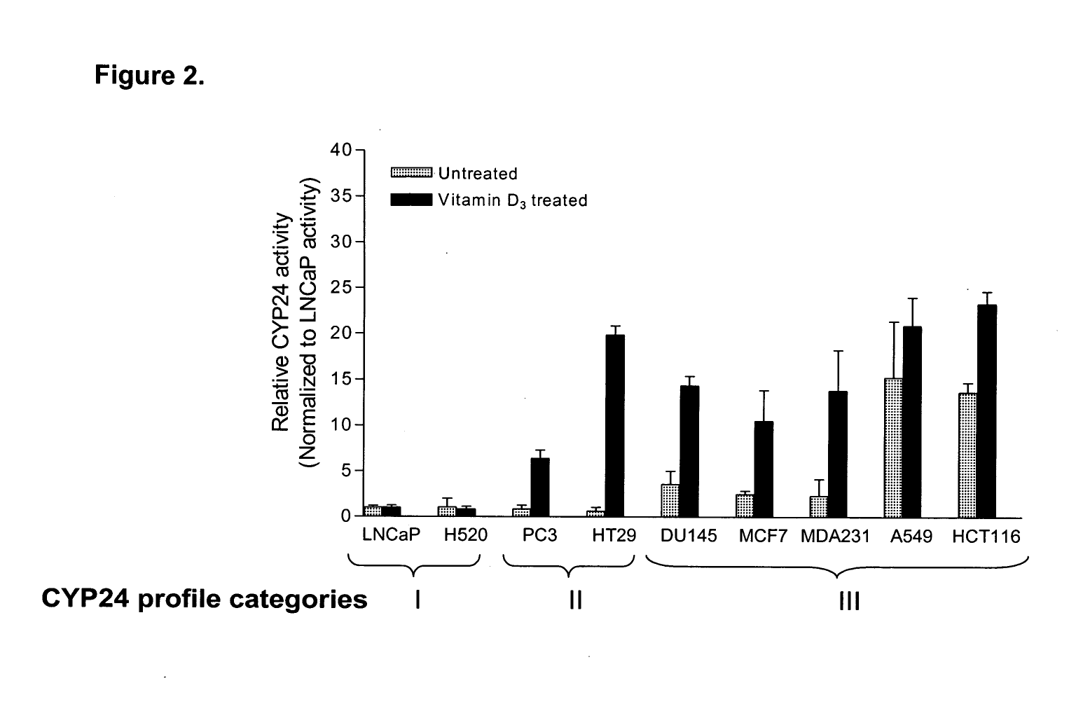

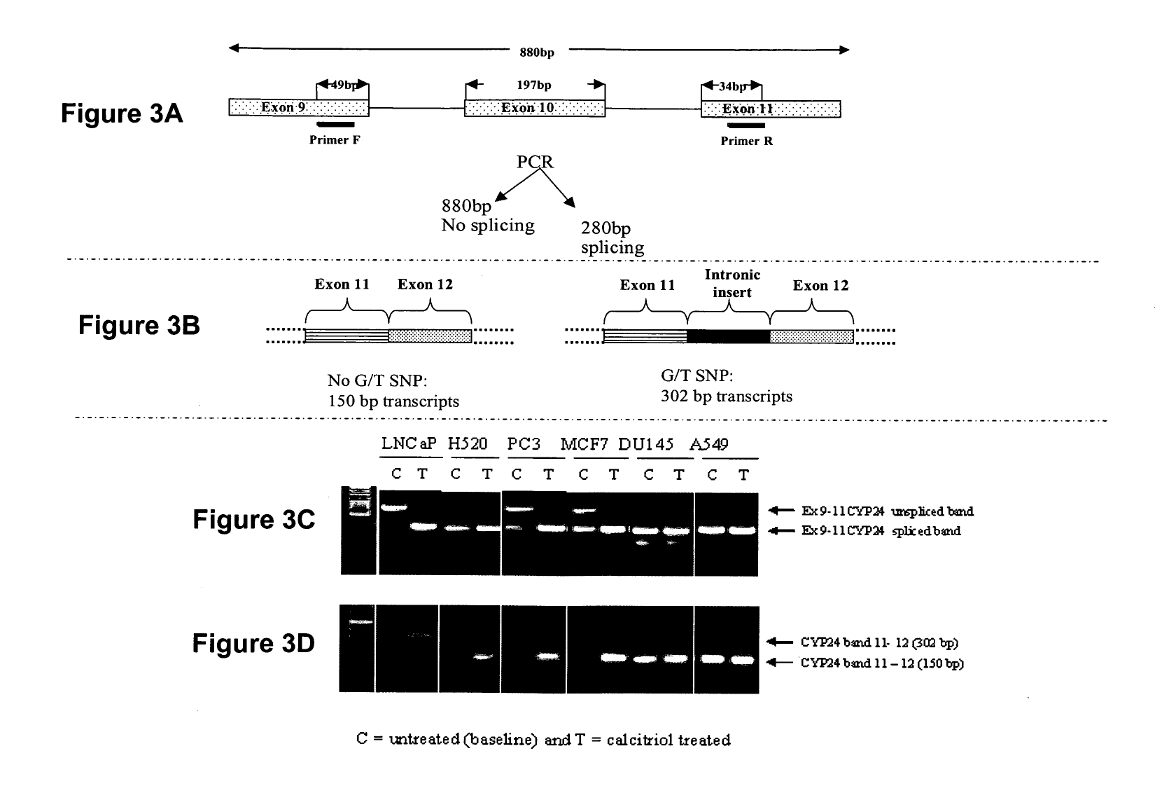

Altered vitamin D metabolism can prolong the biological half-life of calcitriol in circulation and thereby increases exposure, or can result in an individual being resistant to calcitriol. Such changes, over a person's lifetime, are expected to contribute substantially to vitamin D exposure and risk of bone disease, cancer and autoimmune diseases. It is therefore useful to ascertain the presence of the SNPs and/or the splicing pattern of CYP24 mRNA in individuals in need of vitamin D therapy to facilitate customization dosing of calcitriol and related compounds. Optimization of dosing is expected to be of benefit when calcitriol is administered for any purpose, which would include but is not limited to potentiating antitumor activity of chemotherapeutic agents and for osteoporosis therapy. In particular, it is expected that smaller doses of calicitriol could be provided for effective calcitriol treatment in individuals identified by the method of the invention as having reduced calcitriol catabolism, while higher dosing could be used for individuals who have higher calcitriol catabolism (i.e., constitutively active CYP24 protein). For example, smaller dosing could avoid, or at least minimize, hypercalcemic toxicity frequently associated with therapeutic administration of calcitriol. Thus, in one embodiment, the invention provides a method for optimizing calcitriol dosing for individual patients by identifying SNPs that are indicative of aberrant CYP24 mRNA splicing, and/or by identifying CYP24 mRNAs that are aberrantly spliced, wherein such identification is indicative that the individual is likely to have reduced calcitriol catabolism. In particular, identification of the presence of SNP number 4 in Table 2, or aberrantly spliced mRNA as shown for LNCaP in FIG. 2D, is considered to be indicative that the individual has reduced calcitriol catabolism. Identification of a cytosine at SNP number 2 in Table 2 is also indicative that an individual has reduced calcitriol catabolism, as evidenced by the altered calcitriol clearance rates obtained from analysis of patient samples as presented in FIG. 3.

In another embodiment, individuals with high calcitriol catabolism may require or tolerate high doses of calcitriol. “High calcitriol catabolism” is considered to mean calcitriol catabolism that results from constitutively expressed CYP24 mRNA and protein. In this regard, and without intending to be bound by any particular theory, it is considered that, for individuals with normal calcitriol catabolism, CYP24 mRNA is present as unspliced or partially spliced heteronuclear RNA in the absence of calcitriol. The presence of calcitriol however, is believed to induce proper splicing of calcitriol mRNA such that functional CYP24 protein is translated from the properly spliced mRNA. However, some genotypes produce correctly spliced CYP24 mRNA whether or not calcitriol is present, and thus are considered to exhibit calcitriol insensitive splicing. Thus, the presence of predominantly correctly spliced CYP24 mRNA in an individual via calcitriol insensitive splicing is considered to indicate that the individual would benefit from a higher calcitriol dose than a normal individual.

It will be recognized by those skilled in the art that identifying an individual as likely to have altered calcitriol catabolism is also useful for determining dosing regimes for calcitriol precursors, meaning compounds that are metabolized within the body into calcitriol. For example, from a determination that an individual is likely to have reduced calcitriol catabolism, it is expected that smaller doses of vitamin D, or any other calcitriol precursor, could be used to achieve a desired therapeutic effect. Conversely, from a determination that an individual is likely to be insensitive to calcitriol, it is expected that a higher dose of vitamin D, or any other calcitriol precursor, could be used to achieve a desired therapeutic effect.

In another embodiment, identifying an individual as likely to have reduced calcitriol catabolism facilitates design of a dosing regime using a vitamin D analog compound that does not generate as much (i.e. a lesser degree) of a calcemic response as compared to calcitriol when administered to the individual. The phrase “calcemic response” means alterations in calcium metabolism that are caused by biologically active vitamin D compounds when administered to a subject. A calcemic response includes, but is not limited to, elevated calcium concentrations in serum, increased intestinal absorption of dietary calcium, increased urinary calcium excretion, and increased bone calcium mobilization. Examples of vitamin D analog compounds which generate less of a calcemic response than calcitriol include but are not limited to, 1α,25-(OH)₂-24-epi-D₂, 1α,25-(OH)₂-24a-Homo-D₃, 1α,25-(OH)₂-24a-Dihom-o-D₃, 1α,25(OH)₂-19-nor-D₃, and 20-epi-24-homo-1α,25-(OH)₂-D₃.

In another embodiment, identifying an individual as likely to have high calcitriol catabolism facilitates design of a dosing regime using a CYP24 enzyme inhibitor in combination with calcitriol.

Conventional calcitriol dosing parameters are known in the art and are dependant on the age and size of the individual, as well as the reason for calcitriol therapy, such as the type of disease being treated and its stage. For example, in the case of adult dialysis patients, recommended calcitriol doses are provided by the National Kidney Foundation's Kidney Disease Outcome Quality Initiative (“K/DOQI”) guidelines. In one example, for individuals with Stage 4 chronic kidney disease, a suitable dosage is 0.25 mcg/day administered orally. However, for cancer therapy, dosages are typically significantly higher. For instance, in therapy of androgen independent prostate cancer, one example of a suitable calcitriol dosage is 60 mcg/day administered orally (Tiffany et al., J Urol. (2005) Vol. 174(3):888-92). It will be recognized by those skilled in the art that calcitriol therapy may be combined with additional agents, such as chemotherapeutic agents or with calcium, and that optimization of calcitriol dosing in connection with combination therapies is within the scope of the invention.

Diseases which may benefit from customized calcitriol dosing include, but are not limited to: cancers, hyper- and hypo-parathyroidism, diabetes, psoriasis, wound healing, autoimmune diseases, sarcoidosis and tuberculosis, chronic renal disease, vitamin D dependent rickets, fibrogenisis imperfecta ossium, osteitits fibrosa cystica, osteomalacia, osteoporosis, osteopenia, osteosclerosis, renal osteodytrophy, glucocorticoid antagonism, idopathic hypercalcemia, malabsorption syndrome, steatorrhea, tropical sprue, inflammatory bowel disease, ulcerative colitis and Crohn's disease.

To determine if one or more of the SNPs identified herein are present in an individual, a biological sample can be collected from the individual to provide a source of DNA. For example, analysis can be conducted on DNA isolated from cells in a blood sample. However, any biological sample can be used. Further, in addition to information on systemic vitamin D or calcitriol exposure, an individual's ability for regional exposure can also be evaluated. For example, analysis of a bone marrow sample could provide information about vitamin D accumulation/absorption in the bone and thereby lead to predictive information relating to diseases such as osteoporosis.

Detecting the presence of a polymorphism in DNA can be accomplished by a variety of methods including, but not limited to, polymerase chain reaction (PCR), hybridization with allele-specific oligonucleotide probes (Wallace et al. Nucl Acids Res 6:3543-3557 (1978)), including immobilized oligonucleotides (Saiki et al. PNAS USA 86:6230-6234 (1989)) or oligonucleotide arrays (Maskos and Southern Nucl Acids Res 21:2269-2270 (1993)), allele-specific PCR (Newton et al. Nucl Acids Res 17:2503-25 16 (1989)), mismatch-repair detection (MRD) (Faham and Cox Genome Res 5:474-482 (1995)), denaturing-gradient gel electrophoresis (DGGE) (Fisher and Lerman et al. PNAS USA 80:1579-1583 (1983)), single-strand-conformation-polymorphism detection (Orita et al. Genomics 5:874-879 (1983)), chemical (Cotton et al. PNAS USA 85:4397-4401 (1988)) or enzymatic (Youil et al. PNAS USA 92:87-91 (1995)) cleavage of heteroduplex DNA, methods based on allele specific primer extension (Syvanen et al. Genomics 8:684-692 (1990)), genetic bit analysis (GBA) (Nikiforov et al. Nucl Acids Res 22:4167-4175 (1994)), the oligonucleotide-ligation assay (OLA) (Landegren et al. Science 241:1077 (1988)), the allele-specific ligation chain reaction (LCR) (Barrany PNAS USA 88:189-193 (1991)), gap-LCR (Abravaya et al. Nucl Acids Res 23:675-682 (1995)), and radioactive and/or fluorescent DNA sequencing using standard procedures well known in the art.

To determine if CYP24 mRNA is aberrantly spliced, any suitable technique for isolating mRNA and for analyzing the size and/or sequence of the mRNA can be used. Such analytic techniques include but are not limited to Northern blotting, RT-PCR amplification of cDNA and size or sequence analysis of the same, restriction fragment length polymorphism mapping, nucleic acid array analysis, and any other nucleic acid characterization techniques that can be used or adapted to determine whether or not the CYP24 mRNA contains intronic sequences.

In one embodiment, CYP24 mRNA splicing can be measured in cells obtained from an individual both before and after administering calcitriol or a calcitriol precursor and comparing CYP24 mRNA splicing patterns to determine whether the administration of the calcitriol or calcitriol precursor in culture induces aberrant or correct splicing of the CYP24 mRNA. Alternatively, cells can be obtained from an individual, cultured, and tested to determine whether exposure to calcitriol or a calcitriol precursor induces aberrant or correct splicing, wherein aberrant splicing is indicative that the individual has altered vitamin D metabolism. Similarly, to determine whether CYP24 mRNA splicing is calcitriol insensitive, mRNA obtained from an individual before and after administration of calcitriol can be analyzed. Alternatively, cells can be obtained from the individual, cultured, and tested with and without calcitriol to determine whether splicing of CYP24 mRNA is insensitive to calcitriol.

While the present invention is illustrated by way of the following examples, the examples are meant only to illustrate particular embodiments of the present invention and are not meant to be limiting in any way.

### EXAMPLE 1

This Example provides an analysis of CYP24 enzyme activity in untreated and calcitriol-treated human (prostate, breast, lung and colon) cancer cell lines to characterize their capacity to catabolize calcitriol. Three distinct CYP24 enzyme activity profiles were identified, and each of the three prostate cancer cell lines (LNCaP, PC3 and DU145) exhibited different CYP24 enzyme activity profile (FIG. 2).

We have examined carefully the structure of CYP24 in three human cancer cell lines to determine the effect of calcitriol treatment on CYP24 mRNA splicing between exon 9 and 10 and on exon 11-12 size (FIGS. 3A and 3B). PCR analysis shows different patterns of constitutive and calcitriol-induced splicing between exon 9 and 10 and exon 11-12 fragment sizes in the cancer cell lines exhibiting different CYP24 enzyme activity profiles (compare FIG. 2 to FIGS. 3C and 3D). The forward and reverse primers used for detecting splicing between exon 9 and 11 consisted of SEQ ID NO:3 and SEQ ID NO:4, respectively. These data demonstrate that different isoforms of CYP24 protein exist and indicate that variants such as these are generated by aberrant mRNA splicing.

### EXAMPLE 2

This Example demonstrates the some of the effects of calictriol treatment on CYP24 mRNA splicing. To obtain the results presented in this Example, we performed semi-quantitative RT-PCR analysis which revealed two different CYP24 exon 11-12 transcripts based on size, as shown in FIG. 3C, where a low molecular weight transcript (135 bp) and high molecular weight transcript (307 bp) can be seen. Calcitriol treatment (T) modulated the relative expression of the two transcripts differently in the three prostate cancer cell lines. CYP24 enzyme activity is associated with the expression of the lower molecular weight transcript in the three prostate cancer cell lines as shown in Table 3 (D₃=calcitriol), which describes the relationship between CYP24 protein activity phenotypes and exon 11-12 transcript size before (“C” in FIG. 3) and after (“T” in FIG. 3) calcitriol treatment in prostate cancer cell lines. Sequencing studies demonstrated that the difference in transcript sizes is due to a G/T SNP in the splicesome recognition site that causes the insertion of intronic sequences between CYP24 exon 11 and exon 12 ( FIG. 3B).

TABLE 3

| Prostate | | | |
|---|---|---|---|
| cancer | Exon 11-12 size (bp) | | |
| cells | Baseline | D₃ treated | CYP24A1 enzyme activity profiles |
| LNCaP | None | 307 | Negligible: baseline & D₃ inducible |
| PC3 | None | 135 | Negligible baseline & high D₃ inducible |
| DU145 | 135 | 135 | High baseline & D₃ inducible |

### EXAMPLE 3

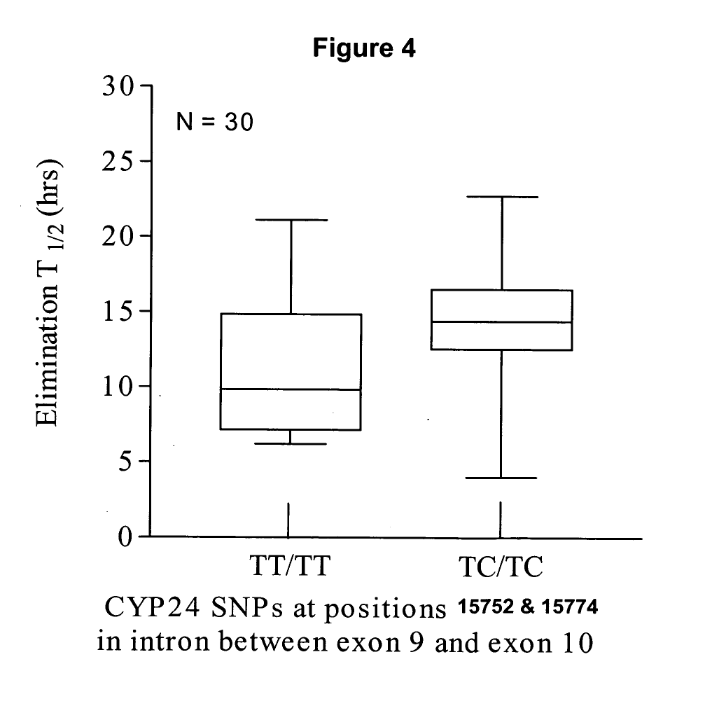

This Example provides an analysis of clinical ramifications of certain CYP24 polymorphisms. To obtain the data presented in this Example, we analyzed CYP24 polymorphisms in the intron between exon 9 and 10 in DNA samples obtained from 30 cancer patients treated with high doses of orally administered calcitriol. The results (FIG. 4) demonstrate that CYP24 polymorphisms were correlated with serum calcitriol elimination half life (T_{1/2}), which is pharmacokinetic measure of systemic calcitriol clearance and thus systemic exposure after calcitriol treatment. (Smith D C, et al., Clin Cancer Res. 1999; 5: 1339-1345).

The data presented in FIG. 4 indicate that a portion of the inter-patient variability in calcitriol exposure is correlated with CYP24 polymorphisms. Calcitriol has recently been shown to potentiate the antitumor activity of docetaxel in a randomized trial in men with advanced prostate cancer. Substantial preclinical data indicate that potentiation of calcitriol relates to dose—thus, according to the results presented herein, exposure to calcitriol in clinical trials can be related to CYP24 polymorphisms and hence, it is expected that efficacy of calcitriol treatment can be ascertained by this readily measured patient characteristic.

The foregoing description of the specific embodiments is for the purpose of illustration and is not to be construed as restrictive. From the teachings of the present invention, those skilled in the art will recognize that various modifications and changes may be made without departing from the spirit of the invention.

We claim:

1. A method for determining whether an individual is likely to have altered calcitriol catabolism, wherein the method comprises:
a) obtaining a biological sample from an individual; and
b) determining the presence or absence of:
i) at least one single nucleotide polymorphisms (SNPs) listed in Table 2;
ii) aberrantly spliced CYP24 mRNA;
iii) calcitriol insensitive splicing; or
iv) combinations of i) through iii);
wherein determining i), ii) or a combination thereof, is indicative that the individual is likely to have reduced calcitriol catabolism relative to an individual who does not have i) or ii), and wherein the presence of iii) is indicative that the individual is likely to have high calcitriol catabolism relative to an individual who does not have iii).

2. The method of claim 1, wherein the aberrant splicing of CYP24 mRNA is induced by administration of calcitriol to the individual prior to obtaining the biological sample from the individual.

3. The method of claim 2, wherein the aberrantly spliced CYP24 mRNA comprises a polynucleotide sequence transcribed from an intron between exon 11 and 12 of the CYP24 gene.

4. The method of claim 1, wherein the at least one SNP is SNP number 4 from Table 2.

5. The method of claim 3, wherein the presence of aberrantly spliced CYP24 mRNA is determined by RT-PCR amplification of the CYP24 mRNA to obtain a CYP24 cDNA and analyzing the size of the cDNA relative to a known size marker to identify a CYP24 cDNA that aberrantly spliced.

6. The method of claim 5, wherein the RT-PCR is performed using a first primer having the sequence of SEQ ID NO:3 and a second primer having the sequence of SEQ ID NO:4, and wherein the RT-PCR amplifies a cDNA comprising a nucleotide sequence from an intron between exon 9 and 10 of the CYP24 gene and/or a nucleotide sequence from an intron between exon 10 and 11 of the CYP24 gene.

7. The method of claim 1, wherein the presence or absence of at least one SNP is determined by PCR amplification of a region of the CYP24 gene comprising a sequence from the intron between exon 9 of the CYP24 gene and exon 10 of the CYP24 gene to obtain a PCR amplification product, and analyzing the sequence of the PCR amplification product to determine the presence or absence of the at least one SNP.

8. The method of claim 1, wherein the presence or absence of at least one SNP is determined by PCR amplification of a region of the CYP24 gene comprising a sequence from the intron between exon 11 of the CYP24 gene and exon 12 of the CYP24 gene to obtain a PCR amplification product, and analyzing the sequence of the PCR amplification product to determine the presence or absence of the at least one SNP.

9. A method for determining, for an individual in need of vitamin D therapy, a dosing regime for calcitriol, a calcitriol precursor, or a vitamin D analog compound, wherein the vitamin D analog compound generates less of a calcemic response than calcitriol, the method comprising:
a) obtaining a biological sample from an individual; and
b) determining in the biological sample the presence or absence of:
i) at least one SNP listed in Table 2;
ii) aberrantly spliced CYP24 mRNA; or
iii) calcitriol insensitive splicing; or
iv) combinations of i) through iii);
wherein the presence of i) or ii), or a combination thereof, is indicative that the individual is a candidate for a lower dose of the calcitriol, the calcitriol precursor, or the vitamin D analog, relative to an individual in need of the vitamin D therapy who does not have i) or ii), and wherein the presence of iii) is indicative that the individual is a candidate for a higher dose of the calcitriol or the calcitriol precursor, relative to an individual in need of the vitamin D therapy who does not have iii).

10. The method of claim 9, wherein the aberrant splicing of CYP24 mRNA is induced by administration of the calcitriol, the calcitriol precursor, or the vitamin D analog, to the individual prior to obtaining the biological sample from the individual.

11. The method of claim 9, wherein the calcitriol precursor is selected from the group consisting of vitamin D and cholecalciferol.

12. The method of claim 9, wherein the vitamin D analog compound is selected from the group consisting of 1α,25-(OH)₂-24-epi-D₂, 1α,25-(OH)₂-24a-Homo-D₃, 1α,25-(OH)₂-24a-Dihom-o-D₃, 1═,25-(OH)₂-19-nor-D₃, and 20-epi-24-homo-1α,25-(OH)₂-D₃

13. The method of claim 9, wherein the aberrantly spliced CYP24 mRNA comprises a polynucleotide sequence transcribed from an intron between exon 9 and 10 of the CYP24 gene or from an intron between exon 11 and 12 of the CYP24 gene.

14. The method of claim 9, wherein the presence of calcitriol insensitive splicing is performed subsequently to administration of calcitriol, a calcitriol calcitriol precursor, or a vitamin D analog to the individual.

15. The method of claim 9, wherein the at least one SNP is SNP number 4 from Table 2.

16. The method of claim 9, wherein the presence of calcitriol insensitive splicing is indicative that the individual is a candidate for therapy with a CYP24 enzyme inhibitor in combination with calcitriol or a calcitriol precursor.

17. The method of claim 9, wherein the presence of aberrantly spliced CYP24 mRNA is determined by RT-PCR amplification of the CYP24 mRNA to obtain a CYP24 cDNA and analyzing the size of the cDNA relative to a known size marker to identify a CYP24 cDNA that aberrantly spliced.

18. The method of claim 17, wherein the RT-PCR is performed using a first primer having the sequence of SEQ ID NO:3 and a second primer having the sequence of SEQ ID NO:4, and wherein the RT-PCR amplifies a cDNA comprising a nucleotide sequence from an intron between exon 9 and 10 of the CYP24 gene and/or a nucleotide sequence from an intron between exon 10 and 11 of the CYP24 gene.
